(19) United States
(12) Patent Application Publication
(10) Pub. No.: US 2011/0060156 A1
(43) Pub. Date: Mar. 10, 2011

(54) Pyruvate Derivatives with Neuroprotective Effect, Process for Preparing the Same and Pharmaceutical Composition Comprising the Same

(75) Inventors: Sung-Hwa Yoon, Suwon-si (KR); Ja-Kyeong Lee, Seoul (KR); Young-Gyun Shin, Suwon-si (KR); Hyun-Ji Kim, Suwon-si (KR)

(73) Assignee: S.H. PHARMA., LTD., Seoul (KR)

(21) Appl. No.: 12/919,951

(22) PCT Filed: Feb. 27, 2009
(86) PCT No.: PCT/KR09/00970

§ 371 Date: Nov. 24, 2010

(30) Foreign Application Priority Data
Feb. 29, 2008 (KR) 10-2008-0018831
Feb. 12, 2009 (KR) 10-2009-0011632

(51) Int. Cl. C07C 69/716; C07C 229/66; C07C 235/80; C07C 65/40

(52) U.S. Cl. 560/174; 562/453; 564/200; 562/463

(57) ABSTRACT

Novel pyruvate derivatives exhibiting outstanding neuroprotective effect, and pharmaceutically acceptable salts thereof, and pharmaceutical compositions for prevention and treatment of brain disease including them as effective ingredient are provided.

### TECHNICAL FIELD

This invention relates to novel compounds for neuroprotection, more particularly to novel pyruvate derivatives capable of preventing cerebral infarction and of maximizing improvement of motor function and recovery from neurological damage, processes for preparing the same, and pharmaceutical compositions comprising the same.

### BACKGROUND ART

Stroke, a major cerebrovascular disease, is the leading cause of death in Korea. The process by which brain cells are damaged following cerebral ischemia involves several processes excessive secretion of excitatory amino acid neurotransmitters in the central nervous system leads to the disruption of normal dynamic balance of calcium level inside and outside the cell due to continued stimulation of the glutamate receptor (NMDA or non-NMDA receptor), thereby resulting in neurotoxicity; nitrogen peroxide (NO) and reactive oxygen species such as oxygen free radical (O₂⁻) produced in excessive during reperfusion results in cell injury; other processes occur in mitochondria.

When ischemia and reperfusion occur in the brain, a delayed damage slowly proceeding for hours to days follows an acute neuronal apoptosis caused by excitatory toxicity. The delayed neuronal cell death is accompanied by an expression of new genes, and is a secondary brain tissue damage process resulting from neuroinflammatory and apoptotic response. A prompt and adequate treatment may reduce the irreversible cell damage (Choi et al., 1992; Lipton et al., 1998).

At present, clinically available drugs for treatment of stroke include thrombolytic drugs such as tissue plasminogen activator (tPA), urokinase, etc., antiplatelet drugs, cerebrovascular dilators, calcium ion channel 1 inhibitors, and the like (Sandercock et al., 1992). They have to be administered within 3 hours of onset of symptoms, or such side effects as nonspecific bleeding, lysis of fibrinogen, or the like are reported (Scheinberg et al., 1994).

A disease involved with many mechanisms, such as stroke, may require the simultaneous administration of one or more medications (combination therapy). Also, development of post-treatment drugs, which provide effect even when treatment is made after a predetermined time following the onset of symptoms, is very important.

Pyruvate is produced mainly by pyruvate kinase at the last stage of glycolysis in cells It is also produced through other metabolic processes such as transamination of alanine. Recently, it was reported that pyruvate not only serves as metabolic intermediate but also performs antioxidative and free radical scavenging actions. The protective mechanisms of pyruvate reported thus far include: (1) role as intermediate of the TCA cycle and metabolic substance; (2) removal of hydrogen peroxide through the process CH₃COCOO—+H₂O₂→CH₃COO—+H₂O+CO₂ (Holleman, 1904); (3) removal of hydroxyl radical [(OH).], one of reactive oxygen species (Dobsak et al., 1999); and (4) inotropic function and sarcoplasmic reticulum ATPase activation. It is also known that iron-mediated oxidative damage plays a crucial role in the pathology evolved in spinal cord contusion injury (SCI). It induces secondary damage in delayed manner and significantly impaired locomotor recovery (Rathore et al., 2008). A growing body of evidence also suggests oxidative stress involvement in neurodegenerative diseases, which includes on Alzheimer disease (AD), Parkinson disease (PD), and amyotrophic lateral sclerosis (ALS).

Cell protective functions provided by pyruvate were researched with regard to various tissue disease conditions. Since the protective effect of intravenous injection of chloropyruvate in acute kidney disease induced by hydrogen peroxide was first identified (Salahudeen et al, 1991), researches demonstrating the protective effect of pyruvate against ischemia-related stress in heart muscle, intestines, liver and the like were reported (Maus et al., 1999; Lee et al., 2001). In addition, protective effect was reported in animal models of cataract induced by galactosemia or diabetes, cerebral ischemia, internal bleeding, and the like.

However, the possibility of pyruvate as a treatment drug is restricted in that (1) pyruvate has a very low solubility in aqueous solution and is very unstable (von Korff, 1964), and (2) it is converted into parapyruvate in aqueous solution and, thereby, acts as a strong inhibitor in the TCA cycle (Montgomery and Webb, 1956).

Because of the aforesaid shortcomings of pyruvate, various pyruvate derivatives have been studied. Among them, ethyl pyruvate (EP) is highly promising as a powerful and effective alternative to pyruvate because of the following advantages. First, as an ester derivative, it is highly lipophilic and has remarkable cell permeation ability. Second, although it has a low solubility in saline or water, the solubility increases markedly in calcium solution (Ringer's solution) (Sims et al., 2001). Third, in calcium solution (Ringer's solution), it is stabilized as it forms anionic enolates as dimmers. Thus, it may serve as a pyruvate precursor. Fourth, ethyl pyruvate is safe. It is approved as a food additive.

Through a preceding research of a stroke animal model (Yu et al., 2005, Stroke), the inventors proposed the possibility of ethyl pyruvate, which showed excellent neuroprotective effect for stroke, as a treatment for stroke, and acquired a patent with regard thereto (Korean Patent Registration No. 10-0686652).

That is, when ethyl pyruvate was abdominally administered within 12 hours after ischemia-reperfusion, the infarct size could be reduced to 50% or less. Further, the infarct size could be reduced by about 20% when treatment was made within 24 hours (Yu et al., 2005). It was confirmed that the infarct reducing effect was accompanied by the recovery of motor function through a rotarod test (Yu et al., 2005). During the process, anti-inflammatory effects of ethyl pyruvate, including inhibition of microglia activation, inhibition of expression of inflammation accelerating cytokines, or the like were observed, and antioxidative action of ethyl pyruvate was confirmed using primarily cultured cells (Kim et al., 2005).

Ethyl pyruvate shows an outstanding neuroprotective effect. In particular, it shows a post-treatment effect excelling all other candidate substances. Ethyl pyruvate is a naturally occurring substance present in cells and is safe as to be approved as food additive. It is expected that the various functions of ethyl pyruvate on top of high cell permeability and stability may be most effectively applied for the diseases involving complex mechanisms, such as stroke.

Meanwhile, aspirin is one of several drugs proved to provide cardiovascular therapeutic and preventive effects. Others include statins, antihypertensives (angiotensin-converting enzyme inhibitors) and hypertension drugs (β-adrenergic blockers).

Aspirin prevents the blocking of blood vessels through irreversible inhibition of platelet aggregation. By irreversibly acetylating cyclooxygenase-1 (COX-1) and cyclooxygenase-2 (COX-2), which strongly induce platelet aggregation and are essential enzymes in the formation of the vasoconstrictor thromboxane A₂ (TXA₂), it inhibits platelet aggregation (Vane, 1971), and, thereby, reduces the embolic size in the central nervous system in ischemic condition and suppresses vasoconstriction. Anti-inflammatory effect through inhibition of NF-κB and neuroprotection through antioxidative effect are also reported. It is also known to reduce damage caused by hypoxia by retarding ATP loss in cells. A low dosage (≧30 mg/day) of aspirin is sufficient for inhibition of TXA₂. However, a higher dose (3-6 g/day) of aspirin is required for neuroprotection through anti-inflammatory or antioxidative actions.

According to a meta-analysis result on the efficacy of aspirin in cardiovascular diseases with respect to its doses, taking 75-325 mg of aspirin every day was effective in treating and preventing cardiovascular diseases in the long term (Hennekens et al., 2006).

Triflusal, which has a similar structure to aspirin, and its metabolite, 2-hydroxy-4-trifluoromethylbenzoic acid (HTB), are used as antiplatelet drugs because they inhibit arachidonic acid metabolism in the platelet and, thereby, prevent platelet aggregation. Triflusal reduces the onset of myocardial infarction in patients with angina pectoris, and relieves pain suffered by patients with peripheral arterial disease. Further, it reduces the incidence of stroke, ischemic heart disease and angionecrosis.

The inventors synthesized various 5-aminosalicylic acid derivatives having a structure similar to that of aspirin. Through experiments, these compounds were identified to provide superior cerebral protective effect and, they were patented in Korea (Korean Patent Registration Nos. 10-0639551 and 10-0751888).

Fluoxetine, represented by the following formula, is fluoxetine hydrochloride developed by Eli Lilly (U.S. Pat. No. 4,018,895). It was approved in 1987 by the Food and Drug Administration (FDA), and is the world's most prescribed antidepressant.

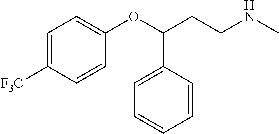

Fluoxetine increases the level of serotonin, a neurotransmitter playing an important role in the modulation of human emotions, in the brain. It has substantially fewer anti-cholinergic adverse effects, such as insomnia, weight increase, vision disorder, cardiac arrhythmia, dry mouth, constipation, and the like, as compared to previous antidepressants, and is taken only once daily. It can be taken without regard to diet and can be administered in combination with most medicines. In addition to depression, it can be effective in treating obsessive-compulsive disorder, bulimia, anthropophobia, kleptomania, post-traumatic stress disorder which is often accompanied by traumatic events, panic disorder with spasmodic symptoms, and the like. Fluoxetine is highly safe.

The production of reactive oxygen species or nitrogen oxide harmful to nerve cells induces apoptosis of the cells. They are reported to be associated with many nerve system diseases such as local and ischemic stroke (Yrjet et al., 1998, 1999; Arvin et al., 2001) and traumatic head injury (Sanches Meijia et al., 2001). According to a research, fluoxetine provides various cerebral neuroprotective effects.

By binding the afore-described antioxidative and anti-inflammatory substances with pyruvate via chemical bondings that can be broken by metabolism, it may be possible to reduce cerebral infarction and to improve in vivo absorption through high solubility in water.

### DISCLOSURE OF INVENTION

### Technical Problem

The inventors have sought to improve solubility in water, increase drug delivery through the blood-brain barrier (BBB), thereby facilitating delivery to the brain, and make the drug administered in the body be degraded into two components by metabolism, which compensate for each other, thereby maximizing the effect of inhibiting cerebral infarction following cerebral ischemia and of improving motor function and recovery from neurological damage, by chemically bonding the drug components exhibiting relative superiority in various damage mechanisms of the nervous system following stroke. As a result, they discovered that the new pyruvate derivatives synthesized by them can prevent damage of brain tissues by inhibiting the activity of microglia and inflammation-inducing cytokines, and completed this invention.

Accordingly, this invention is directed to providing novel pyruvate derivatives, and pharmaceutical compositions for prevention and treatment of brain disease which comprise the novel pyruvate derivatives or pharmaceutically acceptable salts thereof as effective ingredient.

### Technical Solution

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second”, and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

This invention relates to novel compounds providing excellent neuroprotective effect, which are represented by Chemical Formula 1, more particularly to novel pyruvate derivatives and pharmaceutically acceptable salts thereof. The invention also relates to pharmaceutical compositions for treatment and prevention of brain disease comprising the pyruvate derivatives represented by Chemical Formula 1 as effective ingredient, which inhibit activity of microglia and inflammation-inducing cytokines, thereby reducing brain tissue damage.

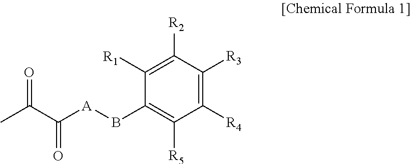

wherein

A represents O, S, NR₁₁ or carbonyl;

B represents a chemical bond or (C1-05)alkylene, wherein the carbon atom of the alkylene may be substituted by one or more of O, S and NR₁₂, and the alkylene may be further substituted by one or more substituent(s) selected from halogen, (C1-C10)alkyl, halo(C1-C10)alkyl, (C1-C10)alkoxy, (C3-C7)cycloalkyl, nitro, amino, mono or di(C1-C10)alkylamino, mono or di(C6-C20)arylamino, (C6-C20)aryl and cyano;

R₁ through R₅ independently represent hydrogen, (C1-C10)alkyl, (C1-C10)alkoxy, (C3-C7)cycloalkyl, (C1-C10)alkoxycarbonyl, (C6-C20)aryloxycarbonyl, (C1-C10)alkylcarbonyl, halogen, cyano, nitro, amino, carboxyl, 2-oxopropanoyloxy, hydroxy, mono or di(C1-C10)alkylamino, mono or di(C6-C20)arylamino or

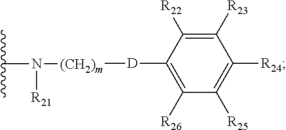

R₁₁ and R₁₂ independently represent hydrogen, (C1-C10)alkyl or (C6-C20)aryl;

D represents a chemical bond, O, NR₃₁ or S;

R₂₁ represents hydrogen, (C1-C10)alkyl or (C6-C20)aryl;

R₂₂ through R₂₆ independently represent hydrogen, (C1-C10)alkyl, (C1-C10)alkoxy, (C3-C7)cycloalkyl, halogen, cyano, nitro, amino, mono or di(C1-C10)alkylamino or mono or di(C6-C20)arylamino;

R₃₁ represents hydrogen, (C1-C10)alkyl or (C6-C20)aryl;

the alkyl, alkoxy and aryl of R₁ through R₅, R₁₁, R₁₂, R₂₁, R₂₂ through R₂₆ and R₃₁ may be further substituted by one or more substituent(s) selected from halogen, (C1-C10)alkyl, halo(C1-C10)alkyl, (C1-C10)alkoxy, cyano, nitro, amino, hydroxy, mono or di(C1-C10)alkylamino and mono or di(C6-C20)arylamino; and

m represents an integer from 1 to 5;

with the proviso that R₁ through R₅ are not hydrogens at the same time.

The pyruvate derivative represented by Chemical Formula 1 according to the present invention may be exemplified by the compounds represented by Chemical Formulas 2 to 4:

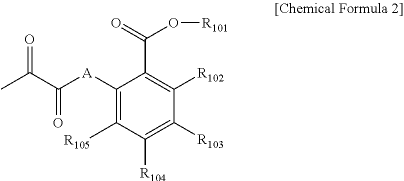

In Chemical Formula 2,

A represents O, S, NR₁₁ or carbonyl;

R₁₁ represents hydrogen, (C1-C10)alkyl or (C6-C20)aryl;

R₁₀₁ represents hydrogen, (C1-C10)alkyl or (C6-C20)aryl;

R₁₀₂ through R₁₀₅ independently represent hydrogen, (C1-C10)alkyl, halo(C1-C10)alkyl, hydroxy(C1-C10)alkyl, (C1-C10)alkoxy, (C3-C7)cycloalkyl, (C1-C10)alkoxycarbonyl, (C6-C20)aryloxycarbonyl, (C1-C10)alkylcarbonyl, halogen, cyano, nitro, amino, carboxyl, 2-oxopropanoyloxy, hydroxy, mono or di(C1-C10)alkylamino, mono or di(C6-C20)arylamino or

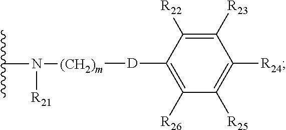

D represents a chemical bond, O, NR₃₁ or S;

R₂₁ represents hydrogen, (C1-C10)alkyl or aryl;

R₂₂ through R₂₆ independently represent hydrogen, (C1-C10)alkyl, halo(C1-C10)alkyl, (C1-C10)alkoxy, (C3-C7)cycloalkyl, halogen, cyano, nitro, amino, mono or di(C1-C10)alkylamino or mono or di(C6-C20)arylamino;

R₃₁ represents hydrogen, (C1-C10)alkyl or (C6-C20)aryl; and

m represents an integer from 1 to 5.

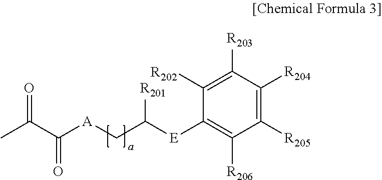

In Chemical Formula 3,

A represents O, S or NR₁₁;

E represents O, NR₁₂ or S;

R₁₁ and R₁₂ independently represent hydrogen, (C1-C10)alkyl or (C6-C20)aryl;

R₂₀₁ represents hydrogen, halogen, (C1-C10)alkyl, halo(C1-C10)alkyl, (C1-C10)alkoxy, (C3-C7)cycloalkyl, nitro, amino, (C6-C20)aryl, mono or di(C1-C10)alkylamino, mono or di(C6-C20)arylamino or cyano;

R₂₀₂ through R₂₀₆ independently represent hydrogen, (C1-C10)alkyl, halo(C1-C10)alkyl, hydroxy(C1-C10)alkyl, (C1-C10)alkoxy, (C3-C7)cycloalkyl, (C1-C10)alkoxycarbonyl, (C6-C20)aryloxycarbonyl, (C1-C10)alkylcarbonyl, halogen, cyano, nitro, amino, carboxyl, 2-oxopropanoyloxy, hydroxy, mono or di(C1-C10)alkylamino, mono or di(C6-C20)arylamino or

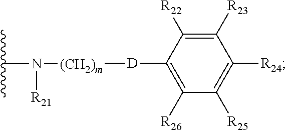

D represents a chemical bond, O, NR₃₁ or S;

R₂₁ represents hydrogen, (C1-C10)alkyl or (C6-C20)aryl;

R₂₂ through R₂₆ independently represent hydrogen, (C1-C10)alkyl, halo(C1-C10)alkyl, (C1-C10)alkoxy, (C3-C7)cycloalkyl, halogen, cyano, nitro, amino, mono or di(C1-C10)alkylamino or mono or di(C6-C20)arylamino;

R₃₁ represents hydrogen, (C1-C10)alkyl or (C6-C20)aryl;

a represents an integer from 1 to 3; and

m represents an integer from 1 to 5.

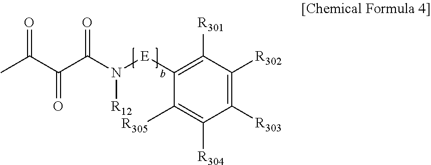

In Chemical Formula 4,

E represents O or S;

R₁₂ represents hydrogen, (C1-C10)alkyl or (C6-C20)aryl;

R₃₀₁ through R₃₀₅ independently represent hydrogen, (C1-C10)alkyl, halo(C1-C10)alkyl, hydroxy(C1-C10)alkyl, (C1-C10)alkoxy, (C3-C7)cycloalkyl, (C1-C10)alkoxycarbonyl, (C6-C20)aryloxycarbonyl, (C1-C10)alkylcarbonyl, halogen, cyano, nitro, amino, carboxyl, 2-oxopropanoyloxy, hydroxy, mono or di(C1-C10)alkylamino, mono or di(C6-C20)arylamino or

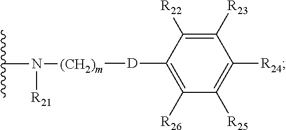

D represents a chemical bond, O, NR₃₁ or S;

R₂₁ represents hydrogen, (C1-C10)alkyl or (C6-C20)aryl;

R₂₂ through R₂₆ independently represent hydrogen, (C1-C10)alkyl, halo(C1-C10)alkyl, (C1-C10)alkoxy, (C3-C7)cycloalkyl, halogen, cyano, nitro, amino, mono or di(C1-C10)alkylamino or mono or di(C6-C20)arylamino;

R₃₁ represents hydrogen, (C1-C10)alkyl or (C6-C20)aryl;

b represents 0 or 1; and

m represents an integer from 1 to 5.

In Chemical Formula 2, A represents O; R₁₀₁ represents hydrogen, methyl or phenyl; R₁₀₂ through R₁₀₅ independently represent hydrogen, methyl, ethyl, n-propyl, i-propyl, n-butyl, i-butyl, t-butyl, n-pentyl, i-pentyl, n-hexyl, n-heptyl, n-octyl, ethylhexyl, trifluoromethyl, hydroxymethyl, hydroxyethyl, methoxy, ethoxy, methoxycarbonyl, phenoxycarbonyl, ethylcarbonyl, chloro, fluoro, cyano, nitro, amino, carboxyl, 2-oxopropanoyloxy, hydroxy or

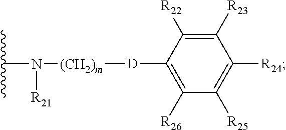

D represents a chemical bond or O; R₂₁ represents hydrogen, methyl or phenyl; R₂₂ through R₂₆ independently represents hydrogen, methyl, trifluoromethyl, methoxy, chloro or fluoro; and m represents an integer from 1 to 5.

In Chemical Formula 3, A represents NR₁₁ or O; E represents O; R₁₁ represents hydrogen, methyl or phenyl; R₂₀₁ represents hydrogen, methyl or phenyl; R₂₀₂ through R₂₀₆ independently represent methyl, ethyl, n-propyl, i-propyl, n-butyl, i-butyl, t-butyl, n-pentyl, i-pentyl, n-hexyl, n-heptyl, n-octyl, ethylhexyl, trifluoromethyl, hydroxymethyl, hydroxyethyl, methoxy, ethoxy, methoxycarbonyl, phenoxycarbonyl, ethylcarbonyl, chloro, fluoro, cyano, nitro, amino, carboxyl, 2-oxopropanoyloxy, hydroxy or

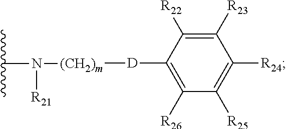

D represents a chemical bond, O or S; R₂₁ represents hydrogen, methyl or phenyl; R₂₂ through R₂₆ independently represent hydrogen, methyl, trifluoromethyl, methoxy, chloro or fluoro; a represents an integer 1 or 2; and m represents an integer from 1 to 5.

In Chemical Formula 4, E represents O; R₁₂ represents hydrogen, methyl or phenyl; R₃₀₁ through R₃₀₅ independently represent hydrogen, methyl, ethyl, n-propyl, i-propyl, n-butyl, i-butyl, t-butyl, n-pentyl, i-pentyl, n-hexyl, n-heptyl, n-octyl, ethylhexyl, triflu-oromethyl, hydroxymethyl, hydroxyethyl, methoxy, ethoxy, methoxycarbonyl, phe-noxycarbonyl, ethylcarbonyl, chloro, fluoro, cyano, nitro, amino, carboxyl, 2-oxopropanoyloxy, hydroxy or

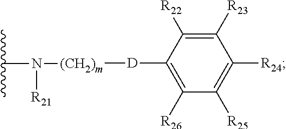

D represents a chemical bond, O or S; R₂₁ represents hydrogen, methyl or phenyl; R₂₂ through R₂₆ independently represent hydrogen, methyl, trifluoromethyl, methoxy, chloro or fluoro; b represents an integer 0 or 1; and m represents an integer from 1 to 5.

Specific examples of the pyruvate derivative according to the present invention include the followings, but are not limited thereto:
2-(2-oxopropanoyloxy)benzoic acid;
2-(2-oxopropanoyloxy)-5-(4-(trifluoromethyl)phenethylamino)benzoic acid;
2-(2-oxopropanoyloxy)-4-(trifluoromethyl)benzoic acid;
2-(2-oxopropanoyloxy)-5-(2,3,5,6-tetrafluoro-4-(trifluoromethyl)benzylamino)benzo is acid;
5-(2-(4-chlorophenoxy)ethylamino)2-(2-oxopropanoyloxy)benzoic acid;
5-(2-(2,4-dichlorophenoxy)ethylamino)-(2-(2-oxopropanoyloxy)benzoic acid;
5-(2-(4-methoxyphenoxy)ethylamino)-2-(2-oxopropanoyloxy)benzoic acid;
2-(2-oxopropanoyloxy)-5-(2-(p-tolyloxy)ethylamino)benzoic acid;
5-(2-(4-fluorophenoxy)ethylamino)-2-(2-oxopropanoyloxy)benzoic acid;
5-(3-(4-fluorophenoxy)propylamino)-2-(2-oxopropanoyloxy)benzoic acid;
4-chloro-2-(2-oxopropanoyloxy)benzoic acid;
4-methoxy-2-(2-oxopropanoyloxy)benzoic acid;
4-hydroxy-2-(2-oxopropanoyloxy)benzoic acid;
4-hydroxy-2,6-bis(2-oxopropanoyloxy)benzoic acid;
2,4,6-tris(2-oxopropanoyloxy)benzoic acid;
2,4-bis(2-oxopropanoyloxy)benzoic acid;
N-methyl-2-oxo-N-(3-phenyl-3-(4-(trifluoromethyl)-phenoxy)propyl)propanamide;
2-oxo-N-(3-phenyl-3-(4-(trifluoromethyl)phenoxy)propyl)propanamide;
2-(2,3-dioxobutanamido)-5-(trifluoromethyl)benzoic acid; and
2-(hydroxymethyl)-5-(trifluoromethyl)phenyl 2-oxopropanoate.

The pyruvate derivative represented by Chemical Formula 1 according to the present invention may be prepared by synthesizing 2-oxopropanoyl chloride from the starting material ethyl pyruvate and then reacting it with a variety of benzene derivatives, as illustrated in Scheme 1. But, the method of preparing the pyruvate derivative represented by Chemical Formula 1 according to the present invention is not restricted thereto. Those skilled in the art will appreciate that the presented preparation method may be modified in various manners.

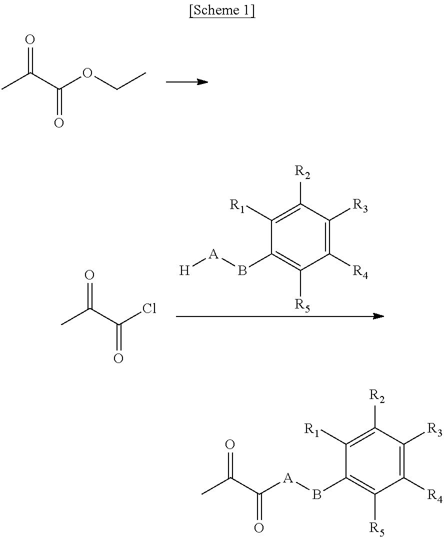

In Scheme 1, A, B, R₁, R₂, R₃, R₄ and R₅ are the same as defined in Chemical Formula 1.

Through an animal model experiment, the pyruvate derivative represented by Chemical Formula 1 according to the present invention was confirmed to provide neuroprotective effect by inhibiting activity of microglia and inflammation-inducing cytokines, thereby preventing brain tissue damage, and to have very high solubility in water.

The pyruvate derivative represented by Chemical Formula 1 according to the present invention is appropriate as an effective ingredient of pharmaceutical compositions for prevention and treatment of brain diseases, such as stroke, ischemic brain disease, paralysis, dementia, Alzheimer's disease, Parkinson's disease, Huntington's disease, epilepsy, Pick's disease, Creutzfeldt-Jakob disease, spinal cord injury, Amyotropic lateral sclerosis, retinal ischemia, memory decline, etc. The pharmaceutically acceptable salts may include organic acid salts and inorganic acid salts. Solvates and hydrates of the salt compounds are also included in the scope of this invention. Pharmaceutically acceptable acid addition salts may be obtained from inorganic acids such as hydrochloric acid, nitric acid, phosphoric acid, sulfuric acid, hydrobromic acid, hydroiodic acid, nitrous acid and phosphorous acid, or from nontoxic organic acids such as aliphatic mono- and dicarboxylates, phenyl-substituted alkanoates, hydroxy-yalkanoates and alkanediates, aromatic acids, and aliphatic and aromatic sulfonates. Examples of the pharmaceutically nontoxic salts include sulfate, pyrosulfate, bisulfate, sulfite, bisulfite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluene-sulfonate, chlorobenzenesulfonate, xylenesulfonate, phenylacetate, phenylpropionate, phenylbutyrate, citrate, lactate, β-hydroxybutyrate, glycolate, malate, tartrate, methane-sulfonate, propanesulfonate, naphthalene-1-sulfonate, naphthalene-2-sulfonate and mandelate. Specifically, hydrochloride may be used.

The dosage of the pyruvate derivative represented by Chemical Formula 1 used to achieve the desired therapeutic effect may be varied depending not only on the amount of the pharmaceutically acceptable salt but also on the particular compound, administration method, subject in need of treatment, and disease to be treated. In general, a dosage of the compound represented by Chemical Formula 1 and the pharmaceutically acceptable salt is from about 1 mg/kg to about 100 mg/kg. The composition may be administered once or several times a day. The dose may be varied depending on the body weight, age, sex and physical conditions of the patient, diet, administration time, administration method, excretion rate, severity of disease, or the like. The composition may be administered orally (pills, capsules, powder or solution) or parenterally (e.g., intravenous administration).

For oral administration, the pharmaceutical composition according to the present invention may be prepared into any existing form, e.g., tablet, powder, dry syrup, chewable tablet, granule, capsule, soft capsule, pill, drink, sublingual tablet, etc.

The composition according to the present invention may be administered to a patient at an effective dose in any bioavailable form. For example, it may be administered orally. The type or method of administration may be selected easily considering the characteristic, stage or other related matters of the disease to be treated. If the composition according to the present invention is in tablet form, it may include one or more pharmaceutically acceptable excipient(s). The content and property of the excipient may be determined depending on the solubility and chemical properties of the selected tablet, the selected administration route, and standard pharmaceutical practices.

In addition to the compound represented by Chemical Formula 1 or its pharmaceutically acceptable salt, the composition according to the present invention may further include one or more pharmaceutically acceptable excipient(s) and therapeutic component(s). The excipient may be a solid or semisolid material that may serve as vehicle or carrier of the active ingredient. Appropriate excipients are well known in the art. The excipient may be selected considering the intended administration method. Specifically, for tablet, powder, chewable tablet, granule, capsule, soft capsule, pill, sublingual tablet or syrup, the therapeutically active drug component may be mixed with a nontoxic and pharmaceutically acceptable inert excipient such as lactose or starch. Optionally, the pharmaceutical tablet may include a binder such as amorphous cellulose, gum tragacanth or gelatin, a disintegrator such as alginic acid, a lubricant such as magnesium stearate, a glidant such as colloidal silicon dioxide, a sweetener such as sucrose or saccharin, or a coloring or flavoring agent such as peppermint or methyl salicylate.

Because of the ease of administration, the tablet may be the desired unit formulation for oral administration. As occasion demands, the tablet may be coated with sugar, shellac or other enteric coating materials, using standard aqueous or non-aqueous techniques.

### ADVANTAGEOUS EFFECTS

The pyruvate derivative according to the invention includes a pyruvate moiety and various antioxidative moieties, e.g. 5-aminosalicylic acid derivative, fluoxetine, etc., in its structure. Therefore, it may be included in pharmaceutical compositions for prevention and treatment of brain disease as an effective ingredient. The pyruvate derivatives included in pharmaceutical compositions as an effective ingredient have very high solubility in water and exhibit increased cell uptake rate, thereby inhibiting activity of microglia and inflammation-inducing cytokines and reducing damage of brain tissues. Further, they exhibit remarkably increased effect of improving motor function and recovery from neurological damage as compared to when the components are administered alone or in combination. Whereas the existing drugs provide no neuroprotective effect 6 hours after the onset of neurological damage and show adverse effects such as nonspecific bleeding, fibrinogen lysis, etc., the novel pyruvate derivative including the pyruvate moiety and the antioxidative moieties, e.g. 5-aminosalicylic acid derivative, fluoxetine, etc., in its structure exhibits high neuroprotective effect even after 6 or 12 hours and is easily administrable because of high solubility in water.

### BRIEF DESCRIPTION OF DRAWINGS

The above and other aspects, features and advantages of the disclosed exemplary embodiments will be more apparent from the following detailed description taken in conjunction with the accompanying drawings in which:

FIG. 1 shows photographs of ischemic brain slices obtained by 2,3,5-triphenyltetrazolium chloride (TTC) staining after the administration of 2-(2-oxopropanoyloxy)benzoic acid, 6 and 12 hours following middle cerebral artery occlusion (MCAD);

FIG. 2 is a graph showing the infarct volume of brain slices depending on the administration dose and administration time of 2-(2-oxopropanoyloxy)benzoic acid;

FIG. 3 shows photographs of ischemic brain slices obtained by TTC staining after the administration of 2-(oxopropanoyloxy)-5-(2,3,5,6-tetrafluoro-4-(trifluoromethyl)benzylamino)benzoic acid prepared in Example 1, 6 and 12 hours following MCAO;

FIG. 4 is a graph showing the infarct volume of brain slices depending on the administration dose and administration time of 2-(oxopropanoyloxy)-5-(2,3,5,6-tetrafluoro-4-(trifluoromethyl)benzylamino)benzoic acid prepared in Example 1;

FIG. 5 shows photographs of ischemic brain slices obtained by TTC staining after the administration of N-methyl-2-oxo-N-(3-phenyl-3-(trifluoromethyl)phenoxy)propyl)propanamide prepared in Example 2, 6 hours following MCAO; and

FIG. 6 shows photographs of ischemic brain slices obtained by TTC staining after the administration of 2-oxo-N-(3-phenyl-3-(4-(trifluoromethyl)phenoxy)propyl)propanamide prepared in Example 3, 6 hours following MCAO.

FIG. 7 is a graph showing the infarct volume of brain slices after the administration of N-methyl-2-oxo-N-(3-phenyl-3-(trifluoromethyl)phenoxy)propyl)propanamide and 2-oxo-N-(3-phenyl-3-(4-(trifluoromethyl)phenoxy)propyl)propanamide prepared in Example 2 and 3, respectively;

FIG. 8 shows photographs of ischemic brain slices obtained by TTC staining after the administration of 2-(2-oxopropanoyloxy)-4-(trifluoromethyl)benzoic acid prepared in Example 4, 6 hours following MCAO.

FIG. 9 is a graph showing the infarct volume of brain slices after the administration of 2-(2-oxopropanoyloxy)-4-(trifluoromethyl)benzoic acid prepared in Example 4.

### MODE FOR THE INVENTION

The examples and experiments will now be described. The following examples and experiments are for illustrative purposes only and not intended to limit the scope of this invention.

### Example 1

### Preparation of 2-(2-oxopropanoyloxy)-5-(2,3,5,6-tetrafluoro-4-(trifluoromethyl)benzylamino)benzoic acid

2-Hydroxy-5-(2,3,5,6-tetrafluoro-4-(trifluoromethyl)benzylamino)benzoic acid (1.00 g, 2.60 mmol) was dissolved using N,N-dimethylformamide (15.0 mL) under nitrogen atmosphere. Potassium carbonate (4.80 g, 13.33 mmol) was added to the solution and, after stirring for 30 minutes, the reaction solution was cooled to 0° C. After adding pyruvoyl chloride (1.22 g, 14.40 mmol), the reaction solution was slowly heated to room temperature and subjected to stirring. After stirring for 4 hours, potassium carbonate was filtered out, and the reaction solvent was removed by concentration under reduced pressure. Stirring was carried out while adding ethyl acetate to the produced oil. The produced solid was filtered and dried under reduced pressure to obtain 2-(2-oxopropanoyloxy)-5-(2,3,5,6-tetrafluoro-4-(trifluoromethyl)benzylamino)benzoic acid (0.74 g, 63.2%).

Melting point: 128° C.; white solid; ¹H NMR (DMSO-d₆) δ 6.907 (m, 3H), 6.270 (t, 1H), 4.416 (s, 2H), 2.496 (d, 2H), 1.659 (s, 3H); ¹³C NMR (DMSO-d₆) δ 168.268, 162.302, 148.474, 146.040, 144.376, 143.643, 142.308, 414.686, 123.538, 120.148, 117.167, 115.643, 108.886, 104.009, 35.898, 23.513.

### Example 2

### Preparation of N-methyl-2-oxo-N-(3-phenyl-3-(4-(trifluoromethyl)phenoxy)propyl)propanamide

Fluoxetine hydrochloride (5.0 g, 14.00 mmol) was dissolved using N,N-dimethylformamide (50.0 mL) under nitrogen atmosphere. Triethylamine (7.31 g, 72.02 mmol) was added to the solution and, after stirring for 30 minutes, the reaction solution was cooled to 0° C. After adding pyruvoyl chloride (3.85 g, 36.14 mmol), the reaction solution was slowly heated to room temperature and subjected to stirring. After stirring for 4 hours, the reaction solvent was removed by concentration under reduced pressure. Ethyl acetate and distilled water were added to the produced oil. The organic layer was washed twice with brine. After drying the organic layer with anhydrous sodium sulfate, ethyl acetate was removed by distillation under reduced pressure. Then, drying was carried out under reduced pressure to obtain N-methyl-2-oxo-N-(3-phenyl-3-(4-(trifluorophenyl)phenoxy)propyl)propanamide (4.74 g, 86.40%).

Transparent oil; ¹H NMR (CDCl₃) δ 7.303 (d, 2H), 7.147 (m, 5H), 6.767 (t, 2H), 5.104 (m, 1H), 3.500 (m, 1H), 3.391 (m, 1H), 2.880 (d, 2H), 2.245 (d, 3H), 2.167 (m, 2H); ¹³C NMR (CDCl₃) δ 198.170, 166.332, 159.825, 139.636, 128.700, 127.911, 126.531, 125.401, 122.906, 115.489, 46.596, 44.609, 37.366, 35.607, 32.702, 29.3638, 27.530.

### Example 3

### Preparation of 2-oxo-N-(3-phenyl-3-(4-(trifluoromethyl)phenoxy)propyl)propanamide

Norfluoxetine (2.06 g, 7.00 mmol) was dissolved using N,N-dimethylformamide (65.0 mL) under nitrogen atmosphere. Potassium carbonate (2.23 g, 21.00 mmol) was added to the solution and, after stirring for 30 minutes, the reaction solution was cooled to 0° C. After adding pyruvoyl chloride (1.45 g, 10.05 mmol), the reaction solution was slowly heated to room temperature and subjected to stirring. After stirring for 4 hours, potassium carbonate was filtered out, and the reaction solvent was removed by concentration under reduced pressure. Ethyl acetate and distilled water were added to the produced oil. The organic layer was washed twice with brine. After drying the organic layer with anhydrous sodium sulfate, ethyl acetate was removed by distillation under reduced pressure. Then, drying was carried out under reduced pressure to obtain 2-oxo-N-(3-phenyl-3-(4-trifluoromethyl)phenoxy)propyl)propanamide (1.51 g, 59.80%).

Transparent oil; ¹H NMR (CDCl₃) δ 7.303 (d, 2H), 3.914 (m, 1H), 3.312 (m, 1H), 2.998 (m, 3H), 2.482 (m, 3H), 2.236 (s, 1H); ¹³C NMR (CDCl₃) δ 198.170, 166.332, 159.825, 139.636, 128.700, 127.911, 126.531, 125.401, 122.906, 115.489, 46.596, 44.609, 37.366, 35.607, 32.702, 29.3638, 27.530.

### Example 4

### Preparation of 2-(2-oxopropanoyloxy)-4-(trifluoromethyl)benzoic acid

2-(2-Oxopropanoyloxy)-4-(trifluoromethyl)benzoic acid was obtained according to the same procedure of Example 1, using 2-hydroxy-4-trifluoromethylbenzoic acid (1.00 g, 4.85 mmol), pyruvoyl chloride (1.55 g, 14.5 mmol) and potassium carbonate (2.00 g, 14.6 mmol).

Melting point: 163° C.; white solid; ¹H NMR (CD₃OD) δ 7.956-7.935 (d, 1H), 7.323-7.316 (s, 2H), 1.805-1.789 (s, 3H); ¹³C NMR (CD₃OD) δ 172.602, 162.690, 158.655, 138.822, 138.625, 131.414, 120.159, 120.121, 119.188, 115.768, 115.730, 106.265, 23.933.

### Example 5

### Preparation of 3-carboxy-4-(2-oxopropanoyloxy)-N-(4-(trifluoromethyl)phenylethyl)benzaminium chloride

5-(Tert-butoxycarbonyl(4-(trifluoromethyl)phenylethyl)amino)-2-hydroxy benzoic acid (600 mg, 1.41 mmol) was dissolved using N,N-dimethylformamide (10.0 mL) under nitrogen atmosphere. Potassium carbonate (584 mg, 4.23 mmol) was added to the solution and, after stirring for 30 minutes, the reaction solution was cooled to 0° C. After adding pyruvoyl chloride (225 mg, 2.11 mmol), the reaction solution was slowly heated to room temperature and subjected to stirring. After stirring for 4 hours, potassium carbonate was filtered out, and the reaction solvent was removed by concentration under reduced pressure. Stirring was carried out while adding ethyl acetate to the produced oil. The produced solid was filtered and dried under reduced pressure to obtain 5-(tert-butoxycarbonyl(4-(trifluoromethyl)phenylethyl)amino)-2-(2-oxopropanyloxy)_{b} enzoic acid. After stirring for 4 hours in 1,4-dioxane 4 N hydrochloric acid, hexane (50 mL) was added, and the produced solid was filtered to obtain 3-carboxy-4-(2-oxopropanyloxy)-N-(4-(trifluoromethyl)phenylethyl)benzaminium chloride (280 mg, 40.1%).

Melting point: 127° C.; white solid; ¹H NMR δ 7.93 (s, 1H), 7.82 (s, 1H), 7.55 (d, 2H) 7.38 (d, 2H), 7.11 (d, 1H), 3.50 (t, 2H), 3.30 (t, 2H), 1.93 (s, 3H).

### Example 6

### Preparation of 4-chloro-2-(2-oxopropanoyloxy)benzoic acid

4-Chloro-2-hydroxybenzoic acid (1.00 g, 6.23 mmol) was dissolved using acetone (30.0 mL). Potassium carbonate (1.72 g, 12.5 mmol) was added to the solution and, after stirring for 1 hour, the reaction solution was cooled to 0° C. After adding pyruvoyl chloride (1.33 g, 12.5 mmol), the reaction solution was slowly heated to room temperature and subjected to stirring. After stirring for 10 minutes, 1 N HCl was added to adjust pH to 4 and extraction was carried out using ethyl acetate. The organic layer was collected and washed with brine. After drying with anhydrous sodium sulfate, the organic layer was subjected to filtration under reduced pressure followed by distillation under reduced pressure. 4-Chloro-2-(2-oxopropanoyloxy)benzoic acid (1.06 g, 70.1%) was obtained as white solid through column chromatography.

White solid; ¹H NMR (CD₃OD) δ 7.797 (d, 1H), 7.126 (m, 2H), 1.831 (s, 3H).

### Example 7

### Preparation of 4-methoxy-2-(2-oxopropanoyloxy)benzoic acid

2-Hydroxy-4-methoxybenzoic acid (1.00 g, 5.95 mmol) was dissolved using acetone (30.0 mL). Potassium carbonate (1.65 g, 11.9 mmol) was added to the solution and, after stirring for 1 hour, the reaction solution was cooled to 0° C. After adding pyruvoyl chloride (1.27 g, 11.9 mmol), the reaction solution was slowly heated to room temperature and subjected to stirring. After stirring for 30 minutes, 1 N HCl was added to adjust pH to 3 and extraction was carried out using ethyl acetate. The organic layer was collected and washed with brine. After drying with anhydrous sodium sulfate, the organic layer was subjected to filtration under reduced pressure followed by distillation under reduced pressure. 4-Methoxy-2-(2-oxopropanoyloxy)benzoic acid (450 mg, 31.8%) was obtained as white solid through column chromatography.

White solid; ¹H NMR (CD₃OD) δ 7.601 (d, 1H), 6.586 (d, 1H), 6.558 (s, 1H), 1.734 (s, 3H).

### Example 8

### Preparation of 4-hydroxy-2-(2-oxopropanoyloxy)benzoic acid

2,4-Dihydroxybenzoic acid (1.00 g, 6.49 mmol) was dissolved using acetone (30.0 mL). Potassium carbonate (3.59 g, 25.9 mmol) was added to the solution and, after stirring for 1 hour, the reaction solution was cooled to 0° C. After adding pyruvoyl chloride (2.77 g, 25.9 mmol), the reaction solution was slowly heated to room temperature and subjected to stirring. After stirring for 1 hour, 1 N HCl was added to adjust pH to 3 and extraction was carried out using ethyl acetate. The organic layer was collected and washed with brine. After drying with anhydrous sodium sulfate, the organic layer was subjected to filtration under reduced pressure followed by distillation under reduced pressure. 4-Hydroxy-2-(2-oxopropanoyloxy)benzoic acid (730 mg, 50.2%) was obtained as white solid through column chromatography.

White solid; ¹H NMR (CD₃OD) δ 7.697 (d, 1H), 6.611 (d, 1H), 6.425 (s, 1H), 1.852 (s, 3H).

### Example 9

### Preparation of 4-hydroxy-2,6-bis(2-oxopropanoyloxy)benzoic acid

2,4,6-Trihydroxybenzoic acid (500 mg, 2.89 mmol) was dissolved using dichloromethane (20.0 mL) and pyridine (1.71 mL). Pyruvoyl chloride (1.21 g, 11.4 mmol) was added after cooling to 0° C. The reaction solution was slowly heated to room temperature and subjected to stirring. After stirring for 12 hours, 1 N HCl was added to adjust pH to 3 and extraction was carried out using ethyl acetate. The organic layer was collected and washed with brine. After drying with anhydrous sodium sulfate, the organic layer was subjected to filtration under reduced pressure followed by distillation under reduced pressure. 4-Hydroxy-2,6-bis(2-oxopropanoyloxy)benzoic acid (612 mg, 68.3%) was obtained as white solid through column chromatography.

White solid; ¹H NMR (CD₃OD) δ 5.994 (s, 1H), 5.943 (s, 1H), 1.794 (s, 6H).

### Example 10

### Preparation of 2,4,6-tris(2-oxopropanoyloxy)benzoic acid

2,4,6-Trihydroxybenzoic acid (500 mg, 2.89 mmol) was dissolved using dichloromethane (20.0 mL) and pyridine (3.42 mL). Pyruvoyl chloride (2.42 g, 22.8 mmol) was added after cooling to 0° C. The reaction solution was slowly heated to room temperature and subjected to stirring. After stirring for 12 hours, 1 N HCl was added to adjust pH to 3 and extraction was carried out using ethyl acetate. The organic layer was collected and washed with brine. After drying with anhydrous sodium sulfate, the organic layer was subjected to filtration under reduced pressure followed by distillation under reduced pressure. 2,4,6-Tris(2-oxopropanoyloxy)benzoic acid (633 mg, 57.6%) was obtained as white solid through column chromatography.

White solid; ¹H NMR (CD₃OD) δ 5.981 (s, 1H), 5.975 (s, 1H), 1.833 (s, 9H).

### Example 11

### Preparation of 2,4-bis(2-oxopropanoyloxy)benzoic acid

2,4-Dihydroxybenzoic acid (1.00 g, 6.29 mmol) was dissolved using dichloromethane (20.0 mL) and pyridine (4.01 mL). Pyruvoyl chloride (3.11 g, 29.2 mmol) was added after cooling to 0° C. The reaction solution was slowly heated to room temperature and subjected to stirring. After stirring for 12 hours, 1 N HCl was added to adjust pH to 3 and extraction was carried out using ethyl acetate. The organic layer was collected and washed with brine. After drying with anhydrous sodium sulfate, the organic layer was subjected to filtration under reduced pressure followed by distillation under reduced pressure. 2,4-Bis(2-oxopropanoyloxy)benzoic acid (812 mg, 43.6%) was obtained as white solid through column chromatography.

White solid; ¹H NMR (CD₃OD) δ 7.892 (d, 1H), 7.079 (s, 1H), 7.040 (d, 1H), 1.785 (s, 6H).

### Example 12

### Preparation of 2-(hydroxymethyl)-5-(trifluoromethyl)phenyl 2-oxopropanoate

2-(2-Oxopropanoyloxy)-4-(trifluoromethyl)benzoic acid (1.50 g, 5.43 mmol) prepared in Example 4 was dissolved using tetrahydrofuran (20.0 mL). After adding boron dimethylsulfite (5.43 ml, 10.9 mmol) dropwise for 10 minutes, the reaction solution was subjected to reflux for 3 hours and then cooled to room temperature. 1 N HCl was added to the reaction solution to adjust pH to 3 and extraction was carried out using ethyl acetate. The organic layer was collected and washed with brine. After drying with anhydrous sodium sulfate, the organic layer was subjected to filtration under reduced pressure followed by distillation under reduced pressure. 2-(Hydroxymethyl)-5-(trifluoromethyl)phenyl 2-oxopropanoate (831 mg, 58.4%) was obtained as white solid through column chromatography.

White solid; ¹H NMR (CDCl₃) δ 10.518 (s, 1H), 8.030 (d, 1H), 7.238 (s, 1H), 7.141 (d, 1H), 2.249 (s, 3H).

### Example 13

### Evaluation of cerebral infarction inhibition effect of 2-(2-oxopropanoyloxy)benzoic acid, 2-(2-oxopropanoyloxy)-5-(2,3,5,6-tetrafluoro-4-(trifluoromethyl)benzylamino)ben zoic acid, N-methyl-2-oxo-N-(3-phenyl-3-(4-(trifluoromethyl)phenoxy)propyl)propanamide, 2-oxo-N-(3-phenyl-3-(4-(trifluoromethyl)phenoxy)propyl)propanamide and 2-(2-oxopropanoyloxy)-4-(trifluoromethyl)benzoic acid

Neuroprotective effect of 2-(2-oxopropanoyloxy)benzoic acid, 2-(2-oxopropanoyloxy)-5-(2,3,5,6-tetrafluoro-4-(trifluoromethyl)benzylamino)benz-oi c acid prepared in Example 1, N-methyl-2-oxo-N-(3-phenyl-3-(4-(trifluoromethyl)phenoxy)propyl)propanamide prepared in Example 2, 2-oxo-N-(3-phenyl-3-(4-(trifluoromethyl)phenoxy)propyl)propanamide prepared in Example 3 and 2-(2-oxopropanoyloxy)-4-(trifluoromethyl)benzoic acid prepared in Example 4 were investigated using an animal model of stroke. A model of focal ischemic stroke in the rat according to the Longa's method (1989) was used. The animal model of stroke was established by occluding the middle cerebral artery (MCA) for 1 hour using nylon suture (middle cerebral artery occlusion, MCAO). In order to investigate the effect of pyruvate derivatives according to the present invention on cerebral ischemia, a rat was decapitated after reperfusion. The whole brain was sliced into 2 mm-thick coronal slices. The slices were immediately stained by immersing in 1% 2,3,5-triphenyl tetrazolium chloride (TTC). After keeping in 4% paraformaldehyde solution at 37° C. for 15 minutes, the brain slices were subjected to measurement and analysis using Quantity One software (Bio-Rad, Hercules, Calif., USA).

(1) Measurement of Infarct Volume for Different Administration Time and Administration Dose of 2-(2-oxopropanoyloxy)benzoic acid

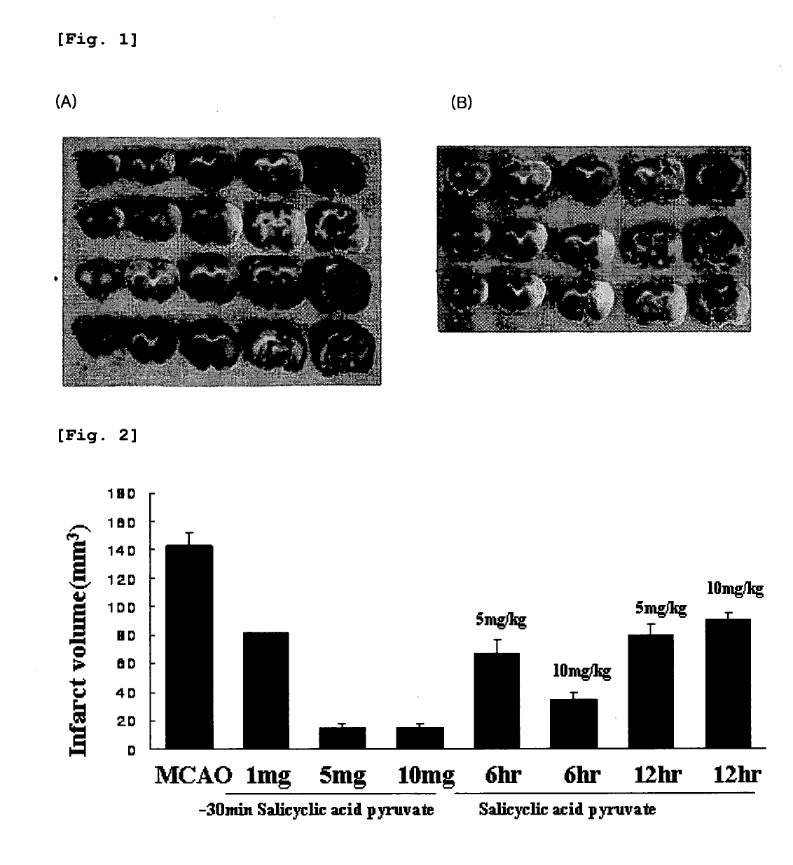

When the animal model of focal ischemic stroke (MCAO) was applied for 1 hour and 2-(2-oxopropanoyloxy)benzoic acid was intravenously administered at a dose of 1, 5 and 10 mg/kg 30 minutes before, the inhibition effect of cerebral infarction was 42.0%, 90.2% and 89.5%, respectively. Therefore, post-treatment experiment was carried out for the doses 1 mg/kg, 5 mg/kg and 10 mg/kg. When intravenous administration was made 6 and 12 hours after reperfusion, there was no change in the infarct volume at a dose of 1 mg/kg. As for 5 mg/kg, treatment after 6 hours reduced the infarct volume to 47.5% as compared to the control group, and treatment after 12 hours reduced the infarct volume to 57.4% as compared to the control group (FIG. 2). When 2-(2-oxopropanoyloxy)benzoic acid was intravenously administered at a dose of 10 mg/kg 6 hours after reperfusion following the application of the animal model of ischemia (MCAO), the infarct volume represented by white color was reduced to 26.6% as compared to the control group (FIG. 1). When intravenous administration was made 12 hours after reperfusion, the infarct volume was reduced to 64.3% as compared to the control group (FIG. 2).

(2) Measurement of Infarct Volume for Different Administration Time and AdMinistration Dose of 2-(2-oxopropanoyloxy)-5-(2,3,5,6-tetrafluoro-4-(trifluoromethyl)benzylamino)benzoic acid

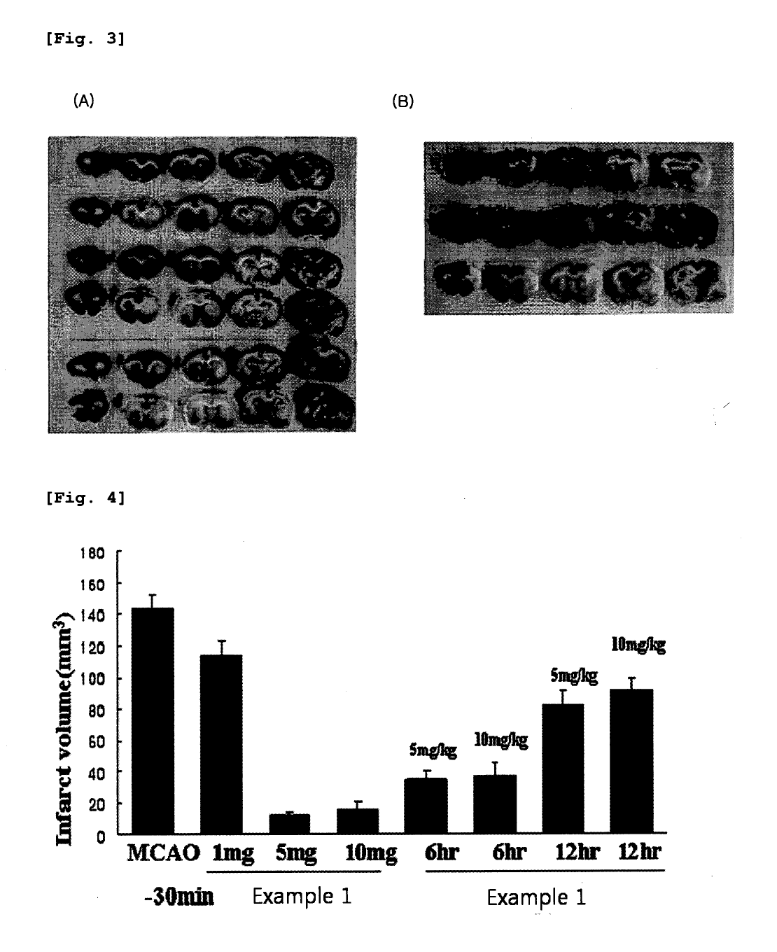

When the animal model of focal ischemic stroke (MCAO) was applied for 1 hour and 2-(2-oxopropanoyloxy)-5-(2,3,5,6-tetrafluoro-4-(trifluoromethyl)benzylamino)benzoic acid was intravenously administered at a dose of 1, 5 and 10 mg/kg 30 minutes before, the inhibition effect of cerebral infarction was 20.2%, 90.9% and 88.4%, respectively. Therefore, post-treatment experiment was carried out for the doses 1 mg/kg, 5 mg/kg and 10 mg/kg. When intravenous administration was made 6 and 12 hours after reperfusion, there was no change in the infarct volume at a dose of 1 mg/kg. As for 5 mg/kg, treatment after 6 hours reduced the infarct volume to 24.5% as compared to the control group, and treatment after 12 hours reduced the infarct volume to 56.8% as compared to the control group (FIG. 4). When 2-(2-oxopropanoyloxy)-5-(2,3,5,6-tetrafluoro-4-(trifluoromethyl)benzylamino)benzoic acid was intravenously administered at a dose of 10 mg/kg 6 hours after reperfusion following the application of the animal model of ischemia (MCAO), the infarct volume represented by white color was reduced to 33.3% as compared to the control group (FIG. 3). When intravenous administration was made 12 hours after reperfusion, the infarct volume was reduced to 62.4% as compared to the control group (FIG. 4).

(3) Measurement of Infarct Volume after the Administration of N-methyl-2-oxo-N-(3-phenyl-3-(4-(trifluoromethyl)phenoxy)propyl)propanamide and 2-oxo-N-(3-phenyl-3-(4-(trifluoromethyl)phenoxy)propyl)propanamide

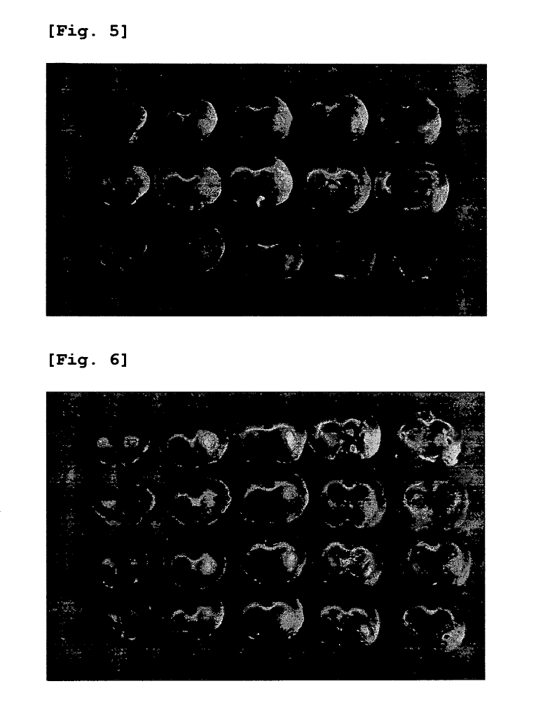

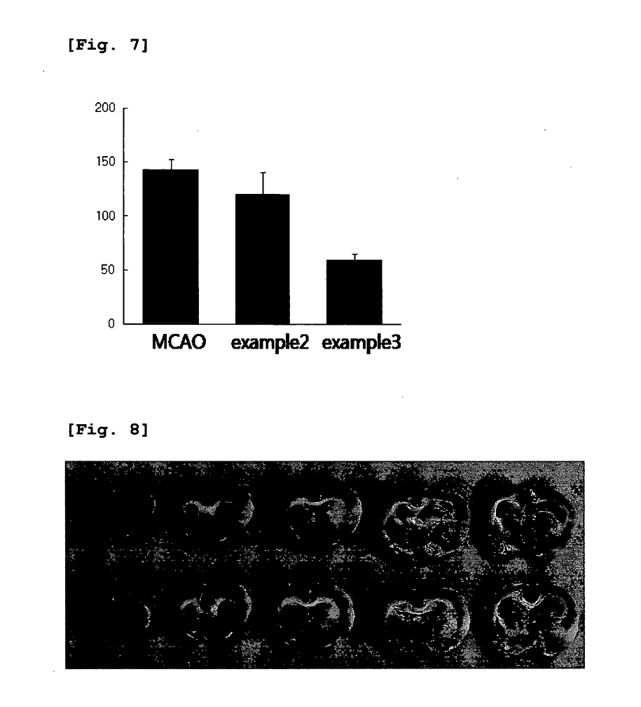

When N-methyl-2-oxo-N-(3-phenyl-3-(4-(trifluoromethyl)phenoxy)propyl) propanamide and 2-oxo-N-(3-phenyl-3-(4-(trifluoromethyl)phenoxy)propyl) propanamide were intravenously administered at a dose of 10 mg/kg 6 hours after reperfusion following the application of the animal model of ischemia (MCAO), the infarct volume represented by white color was reduced to 16.0% and 58.7 as compared to the control group, respectively (FIG. 5, FIG. 6 and FIG. 7).

(4) Measurement of Infarct Volume after the Administration of 2-(2-oxopropanoyloxy)-4-(trifluoromethyl)benzoic acid

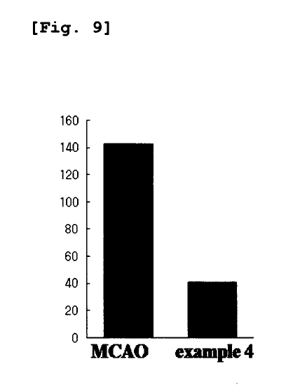

When the animal model of focal ischemic stroke (MCAO) was applied for 1 hour and 2-(2-oxopropanoyloxy)-4-(trifluoromethyl)benzoic acid was intravenously administered at a dose of 5 mg/kg, the inhibition effect of cerebral infarction was 71.3% as compared to the control group (FIG. 8 and FIG. 9).

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

1. A pyruvate derivative represented by Chemical Formula 1 or a pharmaceutically acceptable salt thereof: 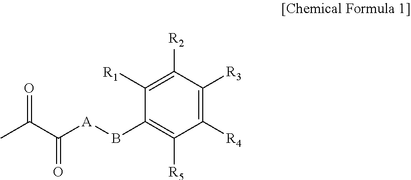
wherein
A represents O, S, NR₁₁ or carbonyl;
B represents a chemical bond or (C1-C5)alkylene, wherein the carbon atom of the alkylene may be substituted by one or more of O, S and NR₁₂, and the alkylene may be further substituted by one or more substituent(s) selected from halogen, (C1-C10)alkyl, halo(C1-C10)alkyl, (C1-C10)alkoxy, (C3-C7)cyclo alkyl, nitro, amino, mono or di(C1-C10)alkylamino, mono or di(C6-C20)arylamino, (C6-C20)aryl and cyano;
R₁ through R₅ independently represent hydrogen, (C1-C10)alkyl, (C1-C10)alkoxy, (C3-C7)cycloalkyl, (C1-C10)alkoxycarbonyl, (C6-C20)aryloxycarbonyl, (C1-C10)alkylcarbonyl, halogen, cyano, nitro, amino, carboxyl, 2-oxopropanoyloxy, hydroxy, mono or di(C1-C10)alkylamino, mono or di(C6-C20)arylamino or 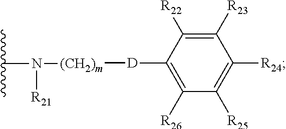
R₁₁ and R₁₂ independently represent hydrogen, (C1-C10)alkyl or (C6-C20)aryl;
D represents a chemical bond, O, NR₃₁ or S;
R₂₁ represents hydrogen, (C1-C10)alkyl or (C6-C20)aryl;
R₂₂ through R₂₆ independently represent hydrogen, (C1-C10)alkyl, (C1-C10)alkoxy, (C3-C7)cycloalkyl, halogen, cyano, nitro, amino, mono or di(C1-C10)alkylamino or mono or di(C6-C20)arylamino;
R₃₁ represents hydrogen, (C1-C10)alkyl or (C6-C20)aryl;
the alkyl, alkoxy and aryl of R₁ through R₅, R₁₁, R₁₂, R₂₁, R₂₂ through R₂₆ and R₃₁ may be further substituted by one or more substituent(s) selected from halogen, (C1-C10)alkyl, halo(C1-C10)alkyl, (C1-C10)alkoxy, cyano, nitro, amino, hydroxy, mono or di(C1-C10)alkylamino and mono or di(C6-C20)arylamino; and
m represents an integer from 1 to 5;
with the proviso that R₁ through R₅ are not hydrogens at the same time.

2. The pyruvate derivative according to claim 1, which is represented by Chemical Formula 2, or a pharmaceutically acceptable salt thereof: 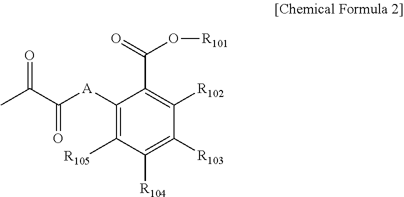
In Chemical Formula 2,
A represents O, S, NR₁₁ or carbonyl;
R₁₁ represents hydrogen, (C1-C10)alkyl or (C6-C20)aryl;
R₁₀₁ represents hydrogen, (C1-C10)alkyl or (C6-C20)aryl;
R₁₀₂ through R₁₀₅ independently represent hydrogen, (C1-C10)alkyl, halo(C1-C10)alkyl, hydroxy(C1-C10)alkyl, (C1-C10)alkoxy, (C3-C7)cycloalkyl, (C1-C10)alkoxycarbonyl, (C6-C20)aryloxycarbonyl, (C1-C10)alkylcarbonyl, halogen, cyano, nitro, amino, carboxyl, 2-oxopropanoyloxy, hydroxy, mono or di(C1-C10)alkylamino, mono or di(C6-C20)arylamino or 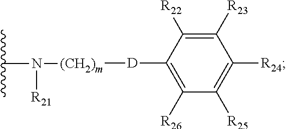
D represents a chemical bond, O, NR₃₁ or S;
R₂₁ represents hydrogen, (C1-C10)alkyl or aryl;
R₂₂ through R₂₆ independently represent hydrogen, (C1-C 10)alkyl, halo(C1-C10)alkyl, (C1-C10)alkoxy, (C3-C7)cycloalkyl, halogen, cyano, nitro, amino, mono or di(C1-C10) alkylamino or mono or di(C6-C20)arylamino;
R₃₁ represents hydrogen, (C1-C10)alkyl or (C6-C20)aryl; and
m represents an integer from 1 to 5.

3. The pyruvate derivative according to claim 1, which is represented by Chemical Formula 3, or a pharmaceutically acceptable salt thereof: 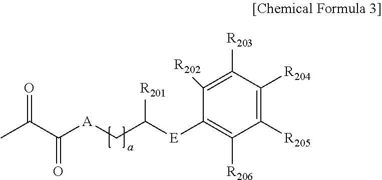
In Chemical Formula 3,
A represents O, S or NR₁₁;
E represents O, NR₁₂ or S;
R₁₁ and R₁₂ independently represent hydrogen, (C1-C10)alkyl or (C6-C20)aryl;
R₂₀₁ represents hydrogen, halogen, (C1-C10)alkyl, halo(C1-C10)alkyl, (C1-C10)alkoxy, (C3-C7)cycloalkyl, nitro, amino, (C6-C20)aryl, mono or di(C1-C10)alkylamino, mono or di(C6-C20)arylamino or cyano;
R₂₀₂ through R₂₀₆ independently represent hydrogen, (C1-C10)alkyl, halo(C1-C10)alkyl, hydroxy(C1-C10)alkyl, (C1-C10)alkoxy, (C3-C7)cycloalkyl, (C1-C10)alkoxycarbonyl, (C6-C20)aryloxycarbonyl, (C1-C10)alkylcarbonyl, halogen, cyano, nitro, amino, carboxyl, 2-oxopropanoyloxy, hydroxy, mono or di(C1-C10)alkylamino, mono or di(C6-C20)arylamino or 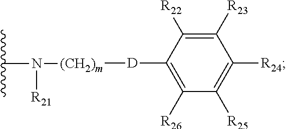
D represents a chemical bond, O, NR₃₁ or S;
R₂₁ represents hydrogen, (C1-C10)alkyl or (C6-C20)aryl;
R₂₂ through R₂₆ independently represent hydrogen, (C1-C10)alkyl, halo(C1-C10)alkyl, (C1-C10)alkoxy, (C3-C7)cyclo alkyl, halogen, cyano, nitro, amino, mono or di(C1-C10)alkylamino or mono or di(C6-C20)arylamino;
R₃₁ represents hydrogen, (C1-C10)alkyl or (C6-C20)aryl;
a represents an integer from 1 to 3; and
m represents an integer from 1 to 5.

4. The pyruvate derivative according to claim 1, which is represented by Chemical Formula 4, or a pharmaceutically acceptable salt thereof: 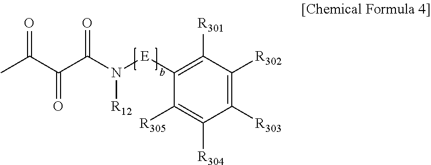
In Chemical Formula 4,
E represents O or S;
R₁₂ represents hydrogen, (C1-C10)alkyl or (C6-C20)aryl;
R₃₀₁ through R₃₀₅ independently represent hydrogen, (C1-C10)alkyl, halo(C1-C10)alkyl, hydroxy(C1-C10)alkyl, (C1-C10)alkoxy, (C3-C7)cycloalkyl, (C1-C10)alkoxycarbonyl, (C6-C20)aryloxycarbonyl, (C1-C10)alkylcarbonyl, halogen, cyano, nitro, amino, carboxyl, 2-oxopropanoyloxy, hydroxy, mono or di(C1-C10)alkylamino, mono or di(C6-C20)arylamino or 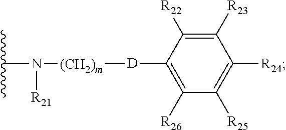
D represents a chemical bond, O, NR₃₁ or S;
R₂₁ represents hydrogen, (C1-C10)alkyl or (C6-C20)aryl;
R₂₂ through R₂₆ independently represent hydrogen, (C1-C10)alkyl, halo(C1-C10)alkyl, (C1-C10)alkoxy, (C3-C7)cycloalkyl, halogen, cyano, nitro, amino, mono or di(C1-C10)alkylamino or mono or di(C6-C20)arylamino;
R₃₁ represents hydrogen, (C1-C10)alkyl or (C6-C20)aryl;
b represents 0 or 1; and
m represents an integer from 1 to 5.

5. The pyruvate derivative according to claim 2 or a pharmaceutically acceptable salt thereof, wherein A represents O; R₁₀₁ independently represents hydrogen, methyl or phenyl; R₁₀₂ through R₁₀₅ independently represent hydrogen, methyl, ethyl, n-propyl, i-propyl, n-butyl, i-butyl, t-butyl, n-pentyl, i-pentyl, n-hexyl, n-heptyl, n-octyl, ethylhexyl, trifluoromethyl, hydroxymethyl, hydroxyethyl, methoxy, ethoxy, methoxycarbonyl, phenoxycarbonyl, ethylcarbonyl, chloro, fluoro, cyano, nitro, amino, carboxyl, 2-oxopropanoyloxy, hydroxy or 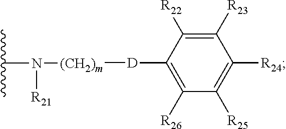
D represents a chemical bond or O; R₂₁ represents hydrogen, methyl or phenyl; R₂₂ through R₂₆ independently represent hydrogen, methyl, trifluoromethyl, methoxy, chloro or fluoro;
and m represents an integer from 1 to 5.

6. The pyruvate derivative according to claim 3 or a pharmaceutically acceptable salt thereof; wherein A represents NR₁₁ or O; E represents O; R₁₁ represents hydrogen, methyl or phenyl; R₂₀₁ represents hydrogen, methyl or phenyl; R₂₀₂ through R₂₀₆ independently represent methyl, ethyl, n-propyl, i-propyl, n-butyl, i-butyl, t-butyl, n-pentyl, i-pentyl, n-hexyl, n-heptyl, n-octyl, ethylhexyl, trifluoromethyl, hydroxymethyl, hydroxyethyl, methoxy, ethoxy, methoxycarbonyl, phenoxycarbonyl, ethylcarbonyl, chloro, fluoro, cyano, nitro, amino, carboxyl, 2-oxopropanoyloxy, hydroxy or 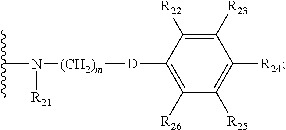
D represents a chemical bond, O or S; R₂₁ represents hydrogen, methyl or phenyl; R₂₂ through R₂₆ independently represent hydrogen, methyl, trifluoromethyl, methoxy, chloro or fluoro; a represents an integer 1 or 2; and m represents an integer from 1 to 5.

7. The pyruvate derivative according to claim 4 or a pharmaceutically acceptable salt thereof; wherein E represents O; R₁₂ represents hydrogen, methyl or phenyl; R₃₀₁ through R₃₀₅ independently represent hydrogen, methyl, ethyl, n-propyl, i-propyl, n-butyl, i-butyl, t-butyl, n-pentyl, i-pentyl, n-hexyl, n-heptyl, n-octyl, ethylhexyl, trifluoromethyl, hydroxymethyl, hydroxyethyl, methoxy, ethoxy, methoxycarbonyl, phenoxycarbonyl, ethylcarbonyl, chloro, fluoro, cyano, nitro, amino, carboxyl, 2-oxopropanoyloxy, hydroxy or 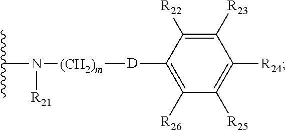
D represents a chemical bond, O or S; R₂₁ represents hydrogen, methyl or phenyl; R₂₂ through R₂₆ independently represent hydrogen, methyl, trifluoromethyl, methoxy, chloro or fluoro; b represents an integer 0 or 1; and m represents an integer from 1 to 5.

8. The pyruvate derivative according to claim 5, which is selected from the following compounds, or a pharmaceutically acceptable salt thereof:
2-(2-oxopropanoyloxy)benzoic acid;
2-(2-oxopropanoyloxy)-5-(4-(trifluoromethyl)phenethylamino)benzoic acid;
2-(2-oxopropanoyloxy)-4-(trifluoromethyl)benzoic acid;
2-(2-oxopropanoyloxy)-5-(2,3,5,6-tetrafluoro-4-(trifluoromethyl)benzylamino)benzoic acid;
5-(2-(4-chlorophenoxy)ethylamino)2-(2-oxopropanoyloxy)benzoic acid;
5-(2-(2,4-dichlorophenoxy)ethylamino)-(2-(2-oxopropanoyloxy)benzoic acid;
5-(2-(4-methoxyphenoxy)ethylamino)-2-(2-oxopropanoyloxy)benzoic acid;
2-(2-oxopropanoyloxy)-5-(2-(p-tolyloxy)ethylamino)benzoic acid;
5-(2-(4-fluorophenoxy)ethylamino)-2-(2-oxopropanoyloxy)benzoic acid;
5-(3-(4-fluorophenoxy)propylamino)-2-(2-oxopropanoyloxy)benzoic acid;
4-chloro-2-(2-oxopropanoyloxy)benzoic acid;
4-methoxy-2-(2-oxopropanoyloxy)benzoic acid;
4-hydroxy-2-(2-oxopropanoyloxy)benzoic acid;
4-hydroxy-2,6-bis(2-oxopropanoyloxy)benzoic acid;
2,4,6-tris(2-oxopropanoyloxy)benzoic acid; and
2,4-bis(2-oxopropanoyloxy)benzoic acid.

9. The pyruvate derivative according to claim 6, which is selected from the following compounds, or a pharmaceutically acceptable salt thereof:
N-methyl-2-oxo-N-(3-phenyl-3-(4-(trifluoromethyl)-phenoxy)propyl)propanamide; and
2-oxo-N-(3-phenyl-3-(4-(trifluoromethyl)phenoxy)propyl)propanamide.

10. The pyruvate derivative according to claim 7, which is 2-(2,3-dioxobutanamido)-5-(trifluoromethyl)benzoic acid, or a pharmaceutically acceptable salt thereof.

11. The pyruvate derivative according to claim 1, which is 22-(hydroxymethyl)-5-(trifluoromethyl)phenyl 2-oxopropanoate, or a pharmaceutically acceptable salt thereof.

12. A pharmaceutical composition for prevention and treatment of brain disease which comprises the pyruvate derivative according to claim 1 or a pharmaceutically acceptable salt thereof as an effective ingredient.

13. The pharmaceutical composition according to claim 12, wherein the brain disease is stroke, ischemic brain disease, paralysis, dementia, Alzheimer's disease, Parkinson's disease, Huntington's disease, epilepsy, Pick's disease, Creutzfeldt-Jakob disease, spinal cord injury, Amyotropic lateral sclerosis, retinal ischemia or memory decline.
